(19) United States
(12) Patent Application Publication
(10) Pub. No.: US 2024/0393314 A1
(43) Pub. Date: Nov. 28, 2024

(54) CLASSIFYING DYNAMIC BEHAVIOR OF FRACTURED RESERVOIRS USING RONALD NELSON'S METHOD AND PRESSURE TRANSIENT ANALYSIS

(71) Applicant: Saudi Arabian Oil Company, Dhahran (SA)

(72) Inventors: Tarek S. AboElsoud, Dhahran (SA); Osman Hamid, Houston, TX (US); Ala Eldeen EL Tayeb Wahballa, Dhahran (SA)

(21) Appl. No.: 18/323,194

(22) Filed: May 24, 2023

(51) Int. Cl. G01N 33/28; E21B 47/06; E21B 47/10; E21B 49/08

(52) U.S. Cl. CPC G01N 33/2823; E21B 47/06; E21B 47/10; E21B 49/08

(57) ABSTRACT

Systems and methods include a computer-implemented method for identifying fractured reservoir types. Well flow data that is received for a well is measured during flowing bottom hole pressure and surface production rates. A transient analysis of the well is performed using the well flow data and analyzed chemical and physical properties of fluid samples of the well. A pressure derivative function is determined for each of multiple different fractured reservoir types. Pattern recognition and fracture type identification are completed based on the transient analysis of the well and using the pressure derivative functions. Field development and production optimization for the well are completed using the pattern recognition and fracture type identification.

### TECHNICAL FIELD

The present disclosure applies to pressure transient analysis (e.g., as part of reservoir testing) of fractured reservoirs as a supporting tool for reservoir characterization, description, and management.

### BACKGROUND

Reservoir engineering techniques typically include the use of pressure transient analysis to study pressure diffusivity behavior of fluid flow inside underground reservoirs, e.g., by creating fluid movement (e.g., drawdowns) followed by fluid stagnation (e.g., pressure build-ups). The pressure variation can be measured over time, with pressure changes plotted versus time on a set of diagnostic plots of pressure and pressure derivatives. The plots can be used to recognize different flow regimes linked to the movement of fluids inside the underground rocks and to estimate the rock permeability (e.g., a measure of rock's ability to allow fluids to flow through porous voids in the rock).

Geology and geo-mechanics are related sciences used in studying subsurface vertical and horizontal stresses that naturally occur due to rock burial and tectonic movements of continental plates. High magnitudes of stress can create subsurface rock failures, which alters the reservoir structure and divides the reservoir structure into segments. Reservoir characterization is hence a method of mapping these failures (called faults or fractures) for the purpose of studying their propagation, abundance, geometry, and intersections by using subsurface seismic imagery and holistic log imaging tools. Pressure transient analysis is a dynamic tool used to integrate with static geological description of the reservoir structure for understanding the fluid flow movement and maximizing the benefits of reservoir fluids production.

### SUMMARY

The present disclosure describes techniques that can be used for classifying fractured reservoirs. In some implementations, a computer-implemented method includes the following. Well flow data that is received for a well is measured during flowing bottom hole pressure and surface production rates. A transient analysis of the well is performed using the well flow data and analyzed chemical and physical properties of fluid samples of the well. A pressure derivative function is determined for each of multiple different fractured reservoir types. Pattern recognition and fracture type identification are completed based on the transient analysis of the well and using the pressure derivative functions. Field development and production optimization for the well are completed using the pattern recognition and fracture type identification.

The previously described implementation is implementable using a computer-implemented method; a non-transitory, computer-readable medium storing computer-readable instructions to perform the computer-implemented method; and a computer-implemented system including a computer memory interoperably coupled with a hardware processor configured to perform the computer-implemented method, the instructions stored on the non-transitory, computer-readable medium.

The subject matter described in this specification can be implemented in particular implementations, so as to realize one or more of the following advantages. The techniques of the present disclosure provide a six to provide a six-type classification of fractured reservoirs' dynamic behavior using Ronald Nelson and pressure transient analysis. The techniques also make it possible to identify two new pressure derivative patterns from pressure transient analysis of non-intersecting natural fractures and depleted discrete intersecting fractures. The techniques of the present disclosure can be integrated with other static reservoir description and characterization data such as fault modeling induced from 3D seismic interpretation, image logs acquired during well logging, and the description of rock cores collected during well drilling. The study of the integrated data can prevent the occurrence of unexpected results that may occur as a result of a limited understanding of fluid movement in hydrocarbon reservoirs. The present disclosure provides an improvement over current techniques by enlarging the scope of pressure transient data interpretation output. Doing this provides a better understanding of all possible outcomes of hydraulic fractures from very low efficiency to very high efficiency. Further, this enables decision makers to optimize on future planning, design, and expenditures of highly-sophisticated and expensive field operations and development, especially for unconventional hydrocarbon reservoirs. Also, the techniques of the present disclosure can help reservoir engineers to improve the hydrocarbon recovery factor by determining the ratio of matrix flow to fracture flow based on classification the new pressure transient. For example, this can lead to avoiding placing new wells into existing natural fractures. Additionally, in case wells were inevitably intersected with fractures, the wells can be recompleted with inflow control devices (ICDs) to eliminate or reduce the flow from the fractures, which in short time will expedite the water early breakthrough.

The details of one or more implementations of the subject matter of this specification are set forth in the Detailed Description, the accompanying drawings, and the claims. Other features, aspects, and advantages of the subject matter will become apparent from the Detailed Description, the claims, and the accompanying drawings.

### DESCRIPTION OF DRAWINGS

FIG. 1A is a diagram showing an example system that includes hydrocarbon flow inside a well and into a production facility during well flow, according to some implementations of the present disclosure.

FIG. 1B is a diagram showing an example system that includes an example of flow stagnation during well shut-in to allow for the pressure to build-up, according to some implementations of the present disclosure.

FIG. 1C includes graphs showing an example of a Cartesian plot of bottom hole pressure and production rates versus time, according to some implementations of the present disclosure.

FIG. 1D shows a derived diagnostic plot of a pressure and pressure derivative function 146 versus time 144 on a log-log scale, according to some implementations of the present disclosure.

FIGS. 2A-2C collectively outline a new classification of fractured reservoirs' dynamic behaviors based on Ronald Nelson's Method, according to some implementations of the present disclosure.

FIG. 2D is a flow diagram showing an example of a workflow for identifying fractured reservoir types, according to some implementations of the present disclosure.

FIGS. 3A-3D collectively show an example of a dynamic response of Type-1 case of a well intersection depleted discrete fracture in a tight matrix, according to some implementations of the present disclosure.

FIGS. 4A-4D collectively show an example of a dynamic response of Type-2 case of a well intersection damaged discrete fracture in permeable rock, according to some implementations of the present disclosure.

FIGS. SA-5D collectively show an example of a dynamic response of Type-3 case of linear discrete fractures with matrix support, according to some implementations of the present disclosure.

FIGS. 6A-6D collectively show an example of a dynamic response of Type-4 case of a fracture network in tight matrix (fractured basement), according to some implementations of the present disclosure.

FIGS. 7A-7D collectively show an example of a dynamic response of Type-5 case of a fracture network in a permeable matrix (double porosity), according to some implementations of the present disclosure.

FIGS. 8A-8D collectively show an example of a dynamic response of Type-6 case of non-intersecting discrete fractures or networks, according to some implementations of the present disclosure.

FIG. 9 is a flowchart of an example of a method for identifying fractured reservoir types, according to some implementations of the present disclosure.

FIG. 10 is a block diagram illustrating an example computer system used to provide computational functionalities associated with described algorithms, methods, functions, processes, flows, and procedures as described in the present disclosure, according to some implementations of the present disclosure.

Like reference numbers and designations in the various drawings indicate like elements.

### DETAILED DESCRIPTION

The following detailed description describes techniques for classifying fractured reservoirs. Various modifications, alterations, and permutations of the disclosed implementations can be made and will be readily apparent to those of ordinary skill in the art, and the general principles defined may be applied to other implementations and applications, without departing from the scope of the disclosure. In some instances, details unnecessary to obtain an understanding of the described subject matter may be omitted so as to not obscure one or more described implementations with unnecessary detail and inasmuch as such details are within the skill of one of ordinary skill in the art. The present disclosure is not intended to be limited to the described or illustrated implementations, but to be accorded the widest scope consistent with the described principles and features.

The present disclosure introduces a new classification of fractured reservoirs dynamic behaviors and provides a new descriptive method to understand fracture abundance and connectivity and their impact on fluid movements inside fractured reservoirs as an upgrade to the currently-existing four-type classification of dynamic behaviors of fractured reservoirs. A new six-type classification of dynamic behaviors of fractured reservoirs using pressure transient analysis is introduced to recognize additional pressure derivative patterns to provide more elaborate descriptions of fluid flow inside fractured reservoirs for both naturally occurring and hydraulically induced fractures.

New classification techniques are introduced to recognize the presence of fractures and to describe their different impact on well productivity or injectivity. The new classification techniques also make it possible to differentiate between intersecting and non-intersection fractures within the wellbore. The pressure transient analysis can be used in combination with numerical simulators to estimate the distance between the well bore and fractures, with the guidance of geological mapping of faults and fractures to emulate realistic situations. The present disclosure also introduces two new pressure derivative patterns:

A first pattern introduced by the present disclosure shows a negative unit slope (−1) followed by a positive quarter slope (+¼) in pressure transient analysis using a pressure derivative log-log plot. This pattern is attributed to a non-intersection conductive fracture located very close to the well. Another previously known pattern was showing negative half slope followed by a positive quarter slope attributed to a non-intersecting conductive fault. Subsidiary patterns show a “negative unit slope (−1) trend followed by either a positive half slope (+½) or positive unit slope (+1)” in relation to low rock porosity and permeability feeding into the non-intersecting conductive fractures.

A second pattern introduced by the present disclosure shows a linear positive half slope (½) followed by a positive linear unit slope (+1) on the pressure derivative log-log plot. This pattern is attributed to a well intersecting a depleted discrete fracture that indicates poor feeding of fluids from the rock into the fracture due to the very low rock permeability. This pattern had been observed in many unconventional tight reservoirs that were hydraulically fracked.

This new classification of fractured reservoirs is an upgrade to Ronald Nelson's method, by being enhanced with pressure transient analysis to elaborate on the description of the dynamic behaviors of fluid flow inside hydrocarbon fractured reservoirs. Two new descriptions of the pressure derivative shapes are added, resulting in the ability to identify six different types of dynamic behaviors with a better understanding of their location, geometry, abundance, hydraulic conductivity, and flow efficiency.

The new classification techniques of the present disclosure provide a better explanation of fractures' impact on well production and production sustainability, which is essential for geologists and reservoir engineers in understanding reservoirs' structural features. As a result, geologists and reservoir engineers can make effective decisions in well placement, completion design, and field development, and apply effective reservoir management for maximum recovery.

Hydraulically fractured reservoirs in unconventional resources can also benefit from the new classification to understand the effectiveness or shortcoming of these fractures in production enhancement in order to optimize future horizontal well drilling design (e.g., including lateral length, number of laterals, angle of inclination, and azimuth) that enables hydraulic fracturing design (e.g., a number of fracturing stages, fractures half length, and fractures interference) to yield sustainable hydrocarbon production with extended drainage area.

The present disclosure introduces new classifications of fractured reservoirs dynamic behaviors. This provides a new descriptive method for understanding fracture abundance and connectivity and their impact on fluid movements inside fractured reservoirs as an upgrade to the previous four-type classification of dynamic behaviors of fractured reservoirs.

Six types of classification of dynamic behaviors of fractured reservoirs can be made using pressure transient analysis. This can facilitate recognizing more additional pressure derivative patterns and provide elaborate descriptions of fluid flow inside fractured reservoirs for both naturally occurring and hydraulically induced fractures.

The additional classifications made possible using techniques of the present disclosure can be used to recognize the presence of fractures and to describe their different impact on well productivity or injectivity. Also, the techniques can be used to differentiate between intersecting and non-intersection fractures with the wellbore. The pressure transient analysis with the help of numerical simulators can estimate the distance between the well bore and fractures with the guidance of geo-mechanical classifications of faults and fracture to emulate realistic situations.

The figure below explains the workflow of data generation from gas and oil wells and the development of diagnostic plots to describe the fluid flow inside these reservoirs and enhancement of hydrocarbon recovery process. Most hydrocarbon reservoirs encounter natural fractures that develop unique dynamic behavior shapes on the diagnostic plots. This new classification of these dynamic behavior provides a better reservoir description/characterization and provide a better enhancement of hydrocarbon recovery.

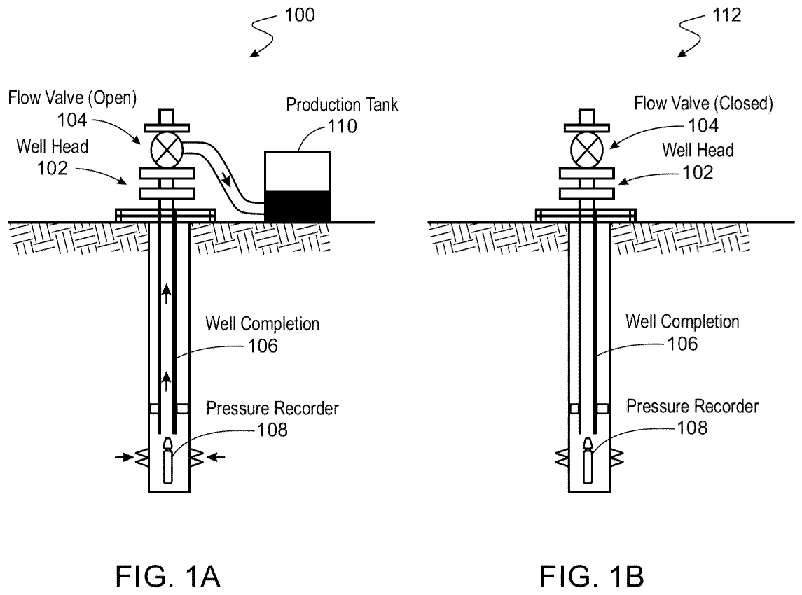

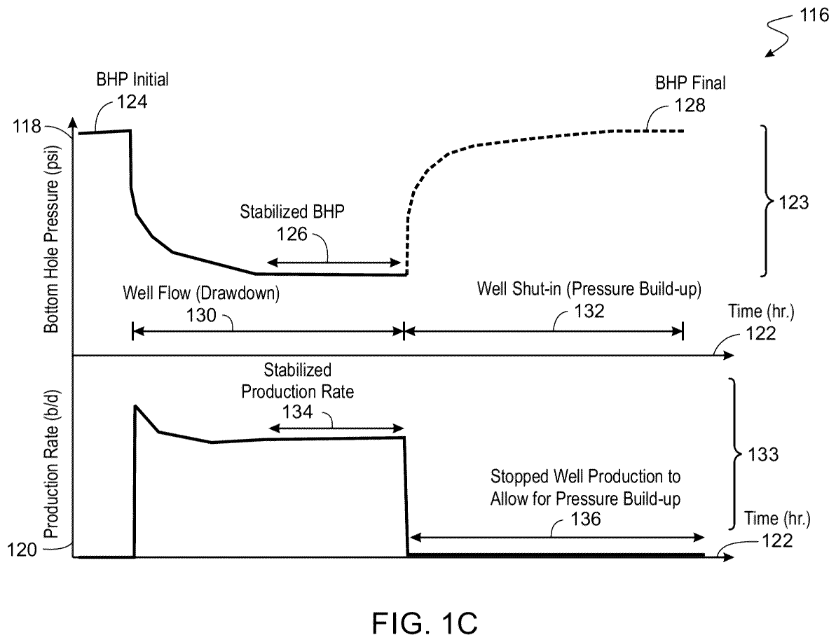

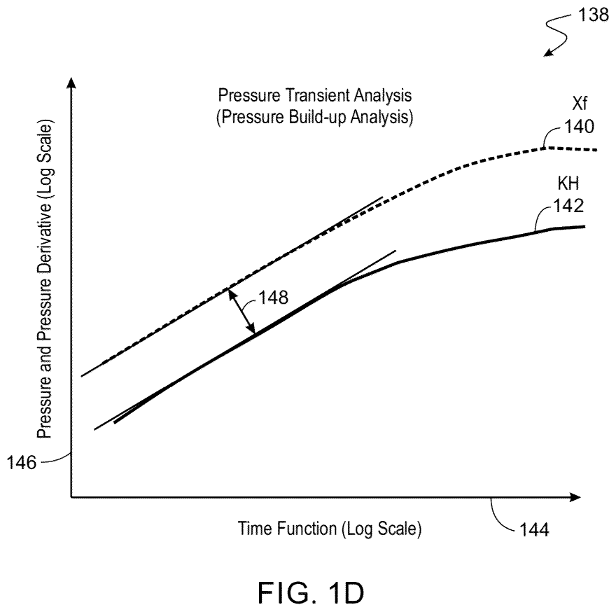

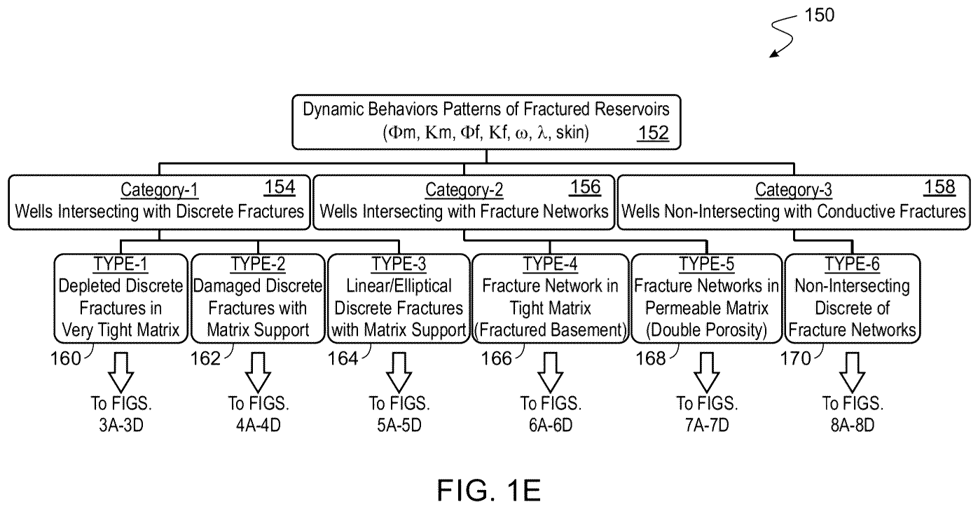

FIG. 1A is a diagram showing an example system 100 that includes hydrocarbon flow inside a well and into a production facility (e.g., a “tank”) during well flow, according to some implementations of the present disclosure. Pressure rates and production rates are recorded versus time. The system 100 includes a well head 102, a flow valve 104 (e.g., depicted in FIG. 1A as being currently open), a well completion 106, a pressure recorder 108, and a production tank 110

FIG. 1B is a diagram showing an example system 112 that includes an example of flow stagnation during well shut-in to allow for the pressure to build-up, according to some implementations of the present disclosure. In the system 112, flow valve 114 is closed. The pressure is recorded versus time.

FIG. 1C includes graphs 116 showing an example of a Cartesian plot of bottom hole pressure and production rates versus time, according to some implementations of the present disclosure. The graphs 116 include Y-axes of a bottom hole pressure (BHP) 118, (e.g., in pounds per square inch (psi)) and a production rate 120 (e.g., in barrels per day (b/d)). Both graphs are plotted relative to an x-axis of time 122 (e.g., in hours (hr.)). A BHP curve 123 includes a BHP initial segment 124, a stabilized BHP segment 126, and a BHP final segment 128, varying over time. The stabilized BHP segment 126 and the BHP final segment 128 are correlated by time to a well flow (drawdown) 130 and a well shut-in (e.g., pressure build-up) 132. A production curve 133 includes a stabilized production 134 and a stopped well production 136 (e.g., stopped to allow for pressure build-up) plotted relative to the production rate 120 over time 122.

FIG. 1D shows a derived diagnostic plot 138 of a pressure and pressure derivative function 146 versus time 144 on a log-log scale, according to some implementations of the present disclosure. The diagnostic plot 138 shows a reservoir rock flow capacity (KH) 142 and fracture half length (X_{f}) 140, with a distance 148 between the curves.

FIG. 1E is a block diagram 150 presenting different dynamic behaviors and classifications of fractured reservoirs, according to some implementations of the present disclosure. The diagram can be used to identify the dynamic behavior type and their properties.

The hierarchy diagram in FIG. 1E represents upgraded classifications of fractured reservoirs' dynamic behaviors 152 divided into three categories 154, 156, and 158, which are subdivided into sub-categories as follows. The Category-1 154 includes wells intersecting discrete fractures, including a Type-1 160 of depleted discrete fractures in very low permeability rock, a Type-2 162 of damaged discrete fractures with matrix support, and a Type-3 164 of linear/elliptical discrete fractures with matrix support. The Category-2 156 includes wells with intersecting fracture networks, including a Type-4 166 of fracture networks in a tight matrix and a Type-5 168 of fracture networks in a permeable matrix. The Category-3 158 includes non-intersecting wells with discrete or fracture networks, including a Type-6 170 of non-intersecting discrete fractures or fracture networks.

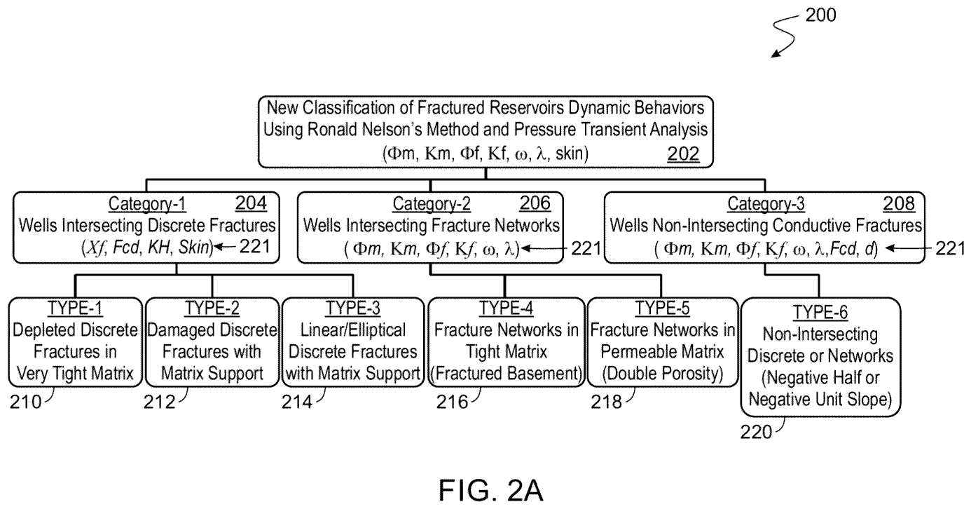

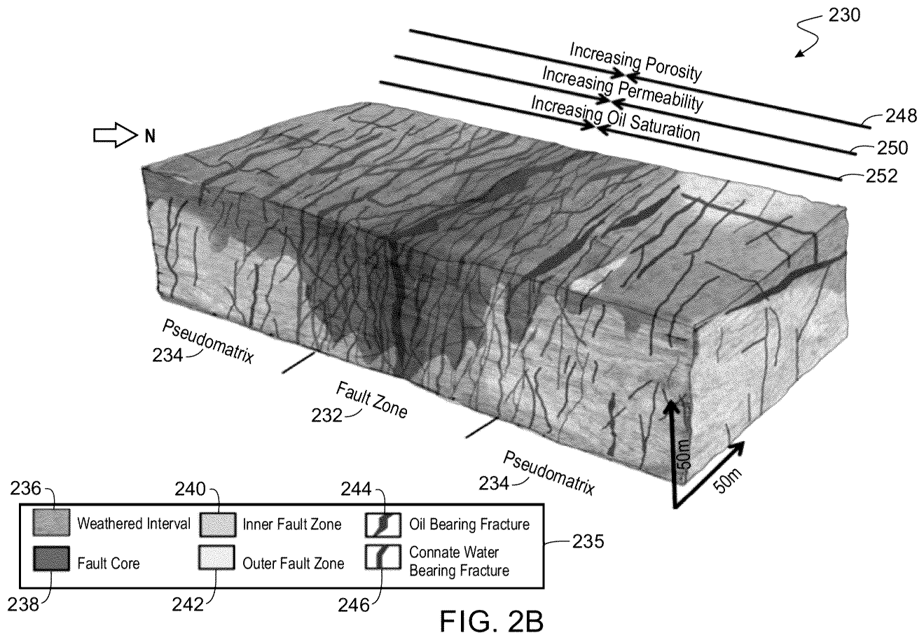

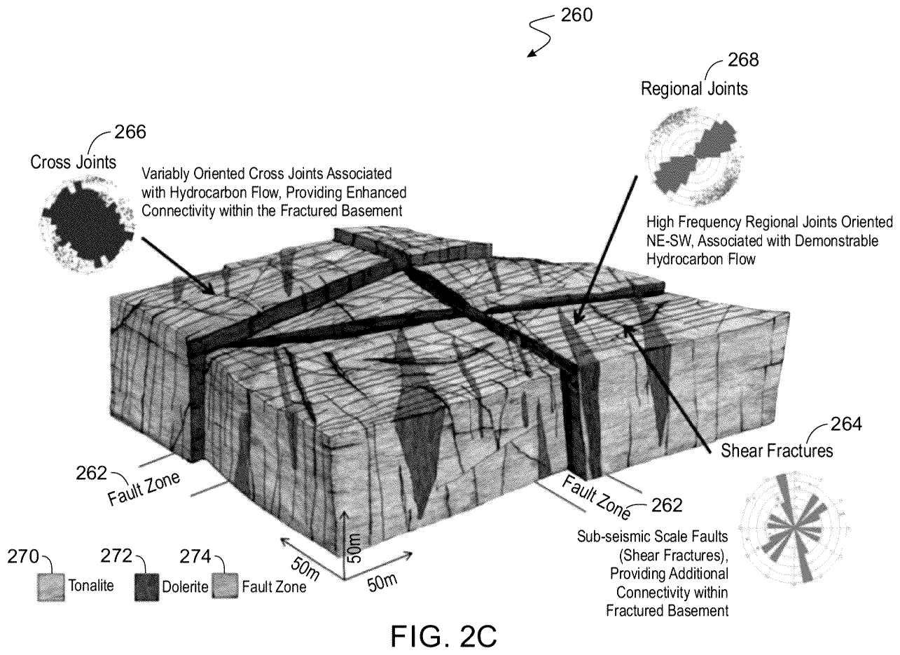

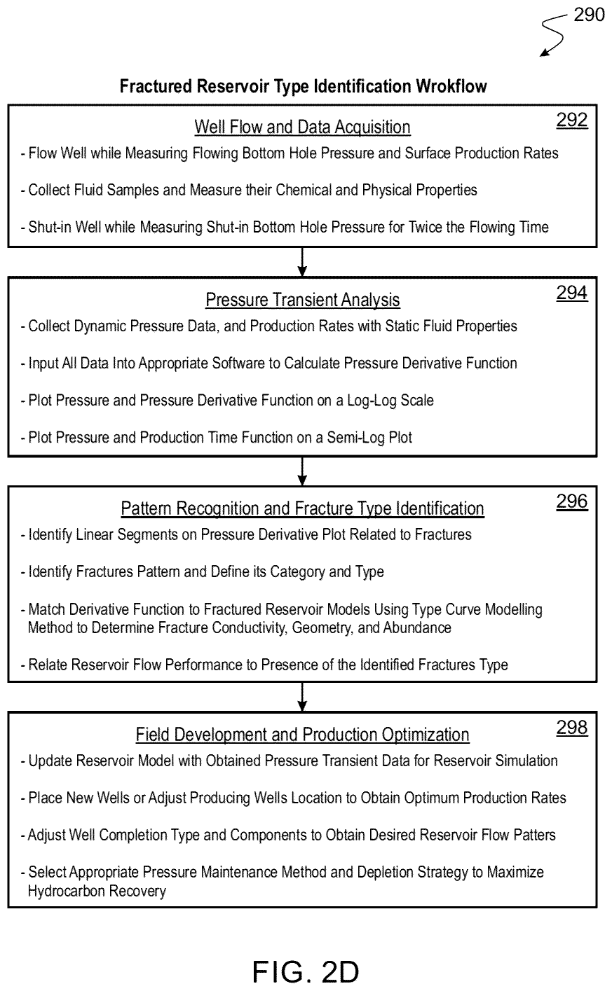

FIGS. 2A-2C collectively outline a new classification of fractured reservoirs' dynamic behaviors based on Ronald Nelson's Method, according to some implementations of the present disclosure. FIG. 2A presents a hierarchy diagram 200 which provides a hierarchy plot and an illustrative graph to explain multiple rock failures with different patterns of faults and fractures and their connectivity, abundance, and apertures. The hierarchy 200 includes the categories and types 202-220 described with reference to FIG. 1E, with the addition of variables 221. The present disclosure is focused on classifying the dynamic behavior of fractures in order to understand their positive or negative impact on reservoir flow mechanisms as an upgrade of Ronald Nelson's method and Kuchuk-Biryukov's classification.

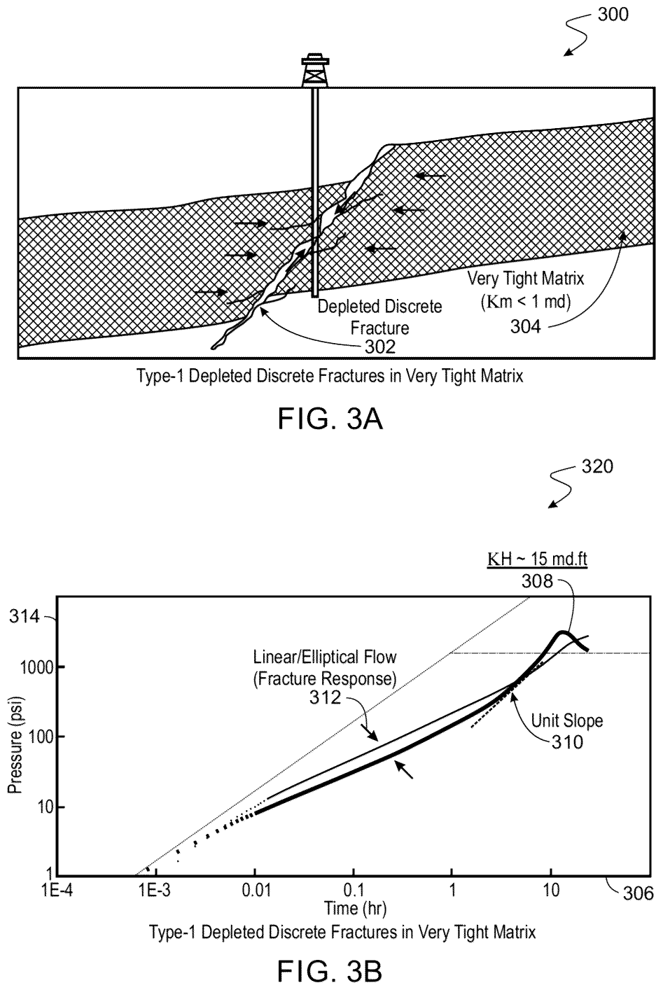

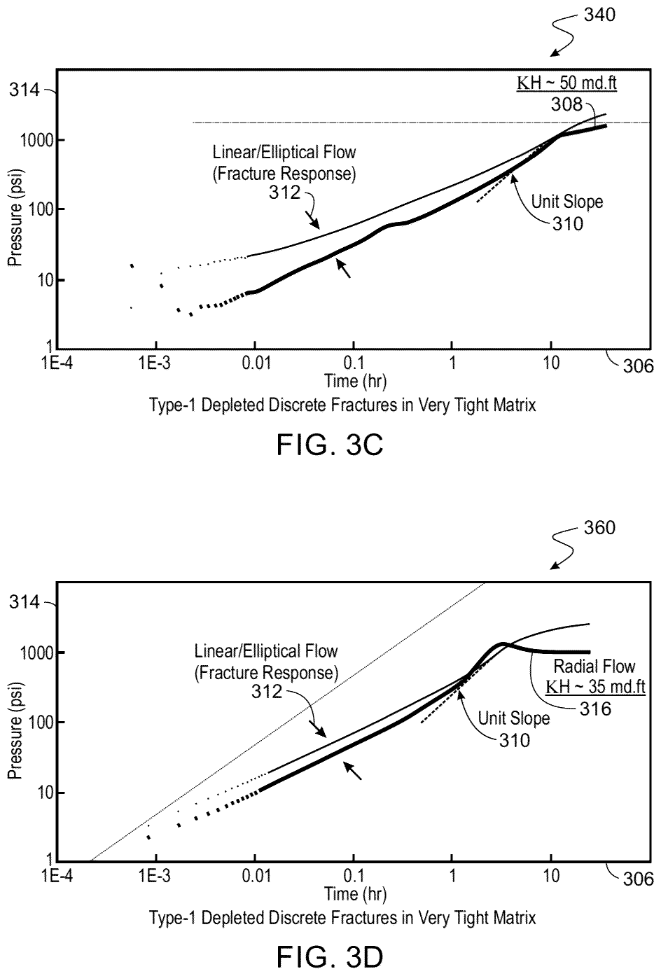

Six patterns of pressure and pressure derivative plots are represented in FIG. 2A. Three patterns are related to the pressure diffusivity in reservoirs with discrete fractures intersecting with the wells. These three patterns are grouped as Category-1. Two pattern types are related to the pressure diffusivity in reservoirs with fracture networks intersecting with the wells. These two patterns are grouped as Category-2. One pattern is related to the pressure diffusivity in reservoirs with non-intersecting discrete or fracture networks. This pattern is defined as Category-3. The hierarchy diagram in FIG. 2A represents the upgraded classification of fractured reservoirs dynamic behaviors divided into three categories, and subdivided the three categories into six different types as presented graphically in FIGS. 3A-8D.

FIGS. 2B-2C collectively present a graphical illustration of the new classification of dynamic behaviors of fractured reservoirs. FIG. 2B illustrates a formation 230 showing examples of a fault zone 232 and pseudo-matrixes 234. A legend 235 identifies different types of geological features 236, 238, 240, 242, 244, and 246. Arrows 248, 250, and 252 identify directions of increasing porosity, permeability, and saturation, respectively.

FIG. 2C illustrates a formation 260 showing examples of fault zones 262, shear fractures 264, cross joints 266, and regional joints 268 of geological features including taconite 270, dolomite 272, and fault zones 274.

FIG. 2D is a flow diagram showing an example of a workflow 290 for identifying fractured reservoir types, according to some implementations of the present disclosure.

At 292, well flow and data acquisition occur. The well is flowed while measuring flowing bottom hole pressure and surface production rates. Fluid samples are collected and their chemical and physical properties are measured. The well is shut-in while measuring shut-in bottom hole pressure for twice the flowing time.

At 294, pressure transient analysis is completed. Dynamic pressure data is collected, and production rates with static fluid properties are collected. The collected data is input into appropriate software to calculate a pressure derivative function. Pressure and pressure derivative functions are plotted on a log-log scale. Pressure and production time functions are plotted on a semi-log plot.

At 296, pattern recognition and fracture type identification are completed. Linear segments related to fractures are identified on the pressure derivative plot. Fractures patterns are identified, and category and type are defined. A derivative function is matched to fractured reservoir models using a type curve modeling method to determine fracture conductivity, geometry, and abundance. Reservoir flow performance is correlated to the presence of the identified fracture types.

At 298, field development and production optimization are completed. The reservoir model is updated with obtained pressure transient data for reservoir simulation. The location of new wells are placed or producing wells are adjusted to obtain optimum production rates. Well completion type and components are adjusted to obtain desired reservoir flow patterns. Appropriate pressure maintenance methods and depletion strategies are selected to maximize hydrocarbon recovery.

FIGS. 3A-3D collectively show an example of a dynamic response of Type-1 case of a well intersection depleted discrete fracture in a tight matrix, according to some implementations of the present disclosure. FIGS. 3A-3D collectively show a graphical drawing to explain the dynamic response of Type-1 case: well intersecting depleted discrete fractures in very low permeability rock. The discrete fractures form either naturally or by hydraulic fracking. During well testing, well flow shows fast decline in pressure and production rate (depletion behavior) with slow pressure recharge (pressure build-up) response during well shut-in. The derivative plot shows the fracture response in the early time followed by a positive unit slope trend followed by late time radial flow. The reservoir rock flow capacity (KH) normally has a very low value. The pressure data was modeled numerically using modern software as a proof of concept and an analytical model needs to be developed in the future to be able to model the pressure behavior.

FIG. 3A shows depleted discrete fractures 302 in a very tight matrix 304 (e.g., permeability Km<1 millidarcy (md)). FIG. 3B shows KH 308 (e.g., approximately 15 millidarcy-feet (md-ft) a unit slope 310, and a linear/elliptical flow 312 (or a fracture response), plotted relative to time 306 and pressure 314 (e.g., in pounds per square inch (psi)). In FIG. 3C, KH 308 is approximately 50 md. In FIG. 3D, a radial flow 316 is approximately 35 md-ft.

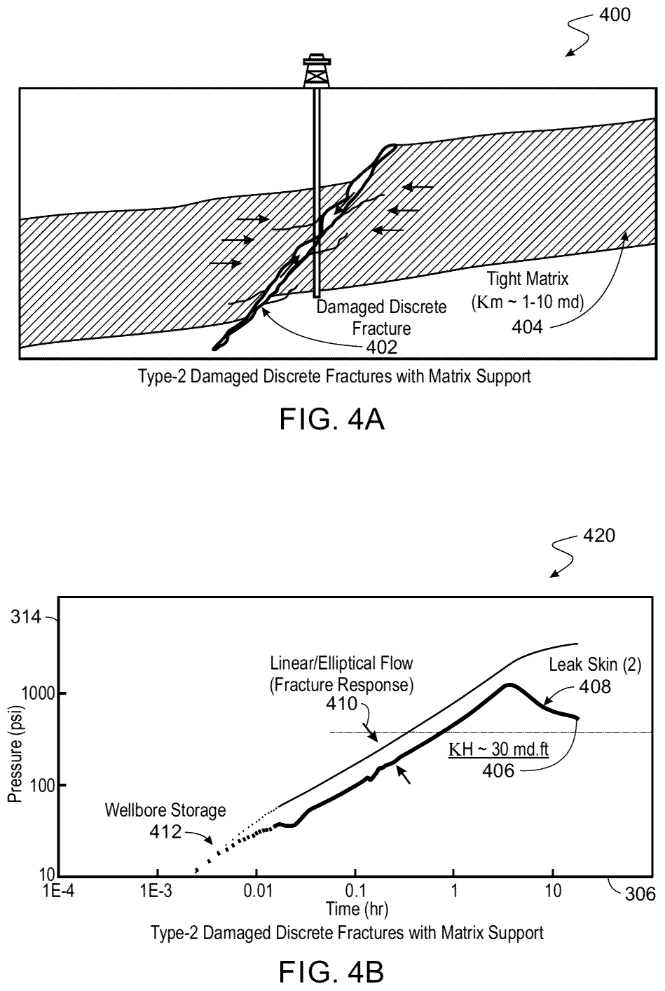

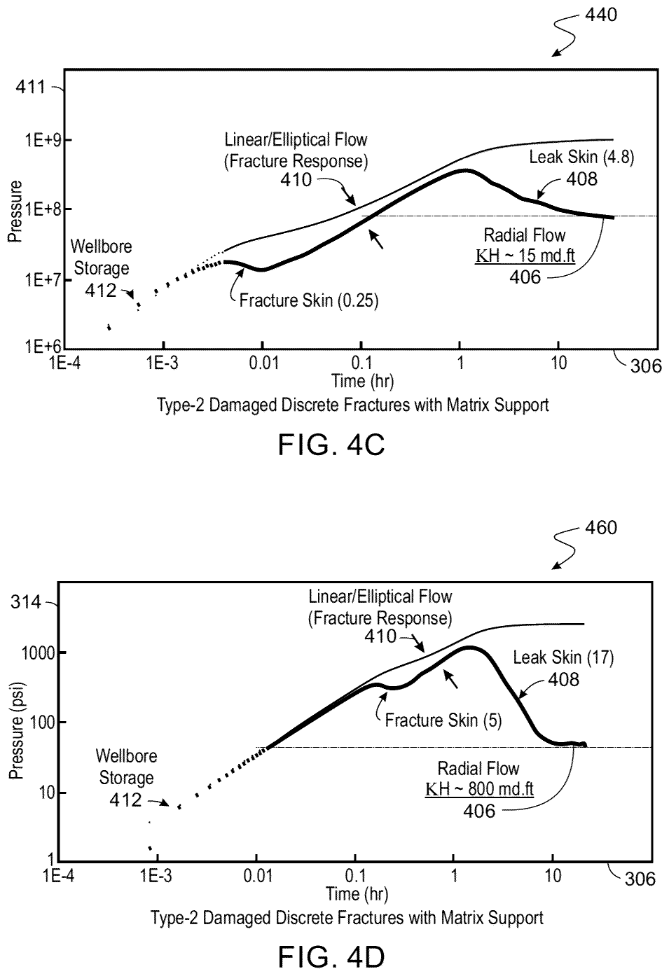

FIGS. 4A-4D collectively show an example of a dynamic response of Type-2 case of a well intersection damaged discrete fracture in permeable rock, according to some implementations of the present disclosure. FIGS. 4A-4D collectively explain the dynamic response of Type-2 case: well intersecting with damaged discrete fractures in permeable rock. These types of fractures can form naturally or by hydraulic fracking. During well testing, well flow shows low production rate but sustainable for a long time. Fracture damage is divided into two types: the first type is called “fracture skin” and the second type is called “leak skin”. The fracture skin happens around the connection between the fracture and well bore due to Darcy or non-Darcy flow. The leak skin happens in the matrix around the fracture face due to rock crushing or due to fracking fluid leak-off during the hydraulic fracking.

Flowing bottom hole pressure drops to low value during well flow and sharp pressure increase is observed during well shut-in. The derivative plot shows the fracture response in the early time followed by a downward trend that is related to “leak skin” or reduced permeability in the region surrounding the fracture. The radial flow in the late time can be used to calculate the KH.

FIG. 4A shows damaged discrete fractures 402 in a tight matrix 404 (e.g., Km in the range of about 1-10 md). FIG. 4B shows a KH 406 (e.g., approximately 30 md-ft), a leak skin 408 of 2 (in dimensionless units) and a linear/elliptical flow 410 (or a fracture response), and wellbore storage 412 plotted relative to time 306 and pressure 314. In FIG. 4C, leak skin 408 is 4.8, KH 406 is approximately 15 md, relative to pressure 411. In FIG. 4D, leak skin 408 is 17,and a radial flow 406 is approximately 800 md-ft.

FIGS. SA-SD collectively show an example of a dynamic response of Type-3 case of linear discrete fractures with matrix support, according to some implementations of the present disclosure. FIGS. SA-5D collectively show the dynamic response of Type-3 case: wells intersecting with discrete fractures in permeable rock. These types of fractures can form naturally or by hydraulic fracking. During well testing, well flow shows a high production rate with long time production sustainability. Both rock porosity/permeability with fractures geometry and conductivity (X_{f}, F_{CD}) contribute to the magnitude of fluid flow inside the reservoir. Flowing bottom hole pressure shows low pressure drawdown during well flow and fast pressure build-up and pressure recharge during well shut-in. The derivative plot shows the fracture response with linear or elliptical flow regimes in the early time followed by a radial flow in the middle time that normally shows good rock permeability.

Elliptical flow regime is also observed on the derivative plot in case of discrete fractures with smaller fracture half-length or lower fracture conductivity (X_{f}, F_{CD}). The derivative plot shows a positive slope varying from 0.625 to 0.875 in the early time.

FIG. 5A shows linear discrete fractures 502 in a permeability matrix 504 (e.g., Km>10 md). FIG. 5B shows a KH 506 (e.g., approximately 220 md-ft), a fracture skin 508 of 0.04, and a linear/elliptical flow 510 (or a fracture response), plotted relative to time 306 and pressure 314. In FIG. 5C, fracture skin 508 is 0.04, KH 506 is approximately 15 md. In FIG. SD, fracture skin 508 is 0.02, and a radial flow 506 is approximately 750 md-ft.

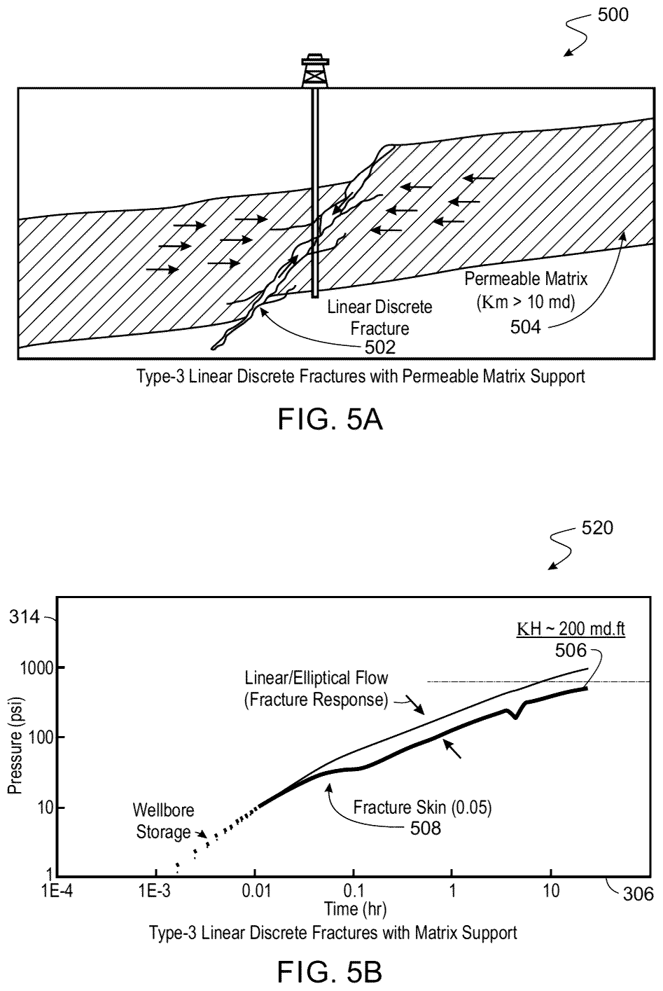

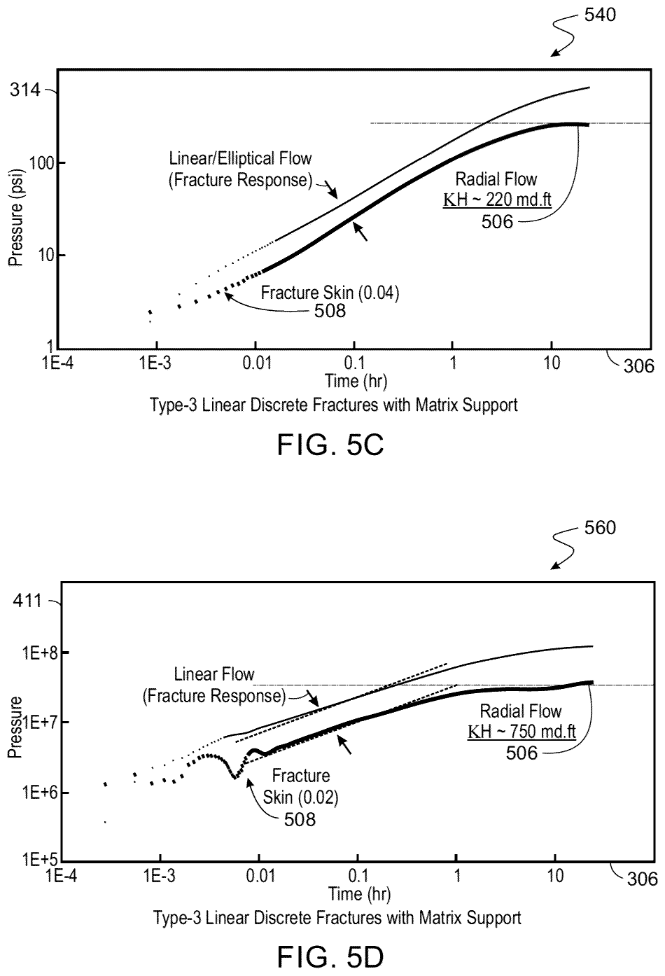

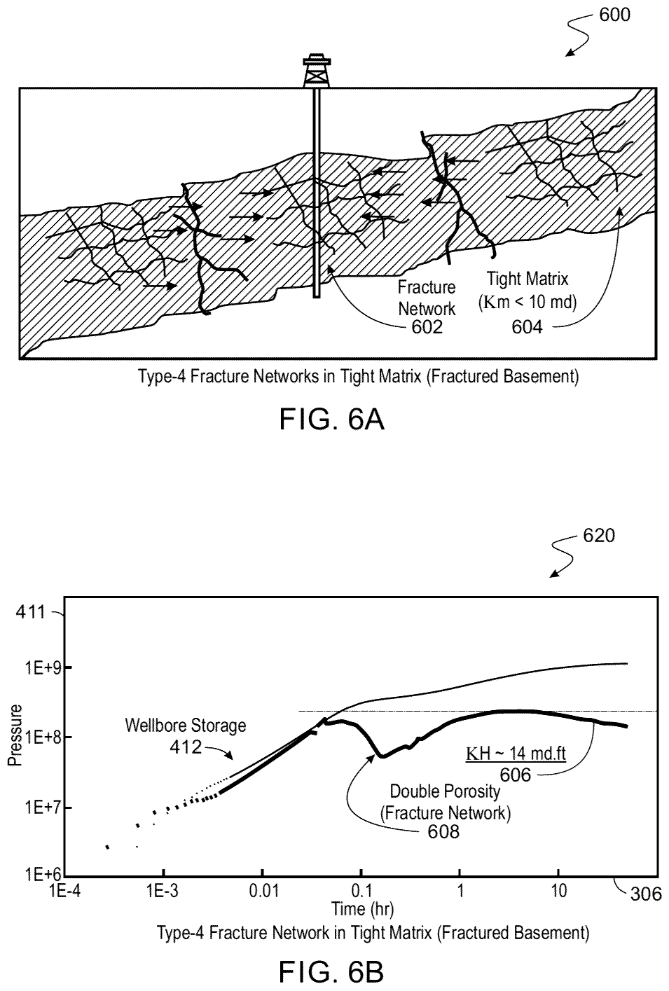

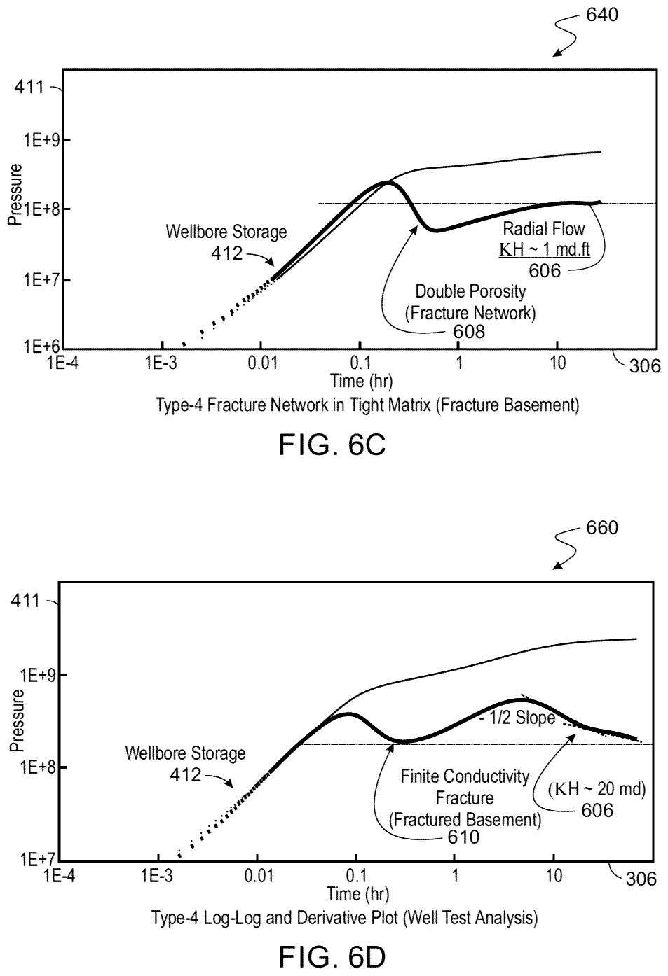

FIGS. 6A-6D collectively show an example of a dynamic response of Type-4 case of a fracture network in tight matrix (fractured basement), according to some implementations of the present disclosure. FIGS. 6A-6D collectively show the dynamic response of Type-4 case: wells intersecting with fracture networks in very low permeability rocks (ex. Fractured Basement). The fracture network is a connected cluster of discrete fractures. Wells drilled to intersect these fracture clusters produce at sustainable low production rates. Fractured basement or tight reservoirs are normally classified as unconventional reservoirs and these reservoirs can only produce if they contain these large connected fractures whether natural or with multi-stage hydraulic fracturing. The derivative plot shows early time “valley shape” followed by a horizontal line (radial flow). The rock porosity/permeability provides the flow support to the fracture network. In this case, the Warren-Root model can be used to calculate the interporosity flow parameter (λ), the storativity coefficient (ω), and the radial flow is used to calculate the matrix quality.

FIG. 6A shows a fracture network 602 in a tight matrix 604 (e.g., permeability Km<10 md). FIG. 6B shows a KH 606 (e.g., approximately 14 md-ft), a double porosity 608, plotted relative to time 306 and pressure 314. In FIG. 6C, double porosity 608, KH 606 is approximately 1 md. FIG. 6D shows a 1½ slope of the KH 606, and a finite conductivity fracture 610.

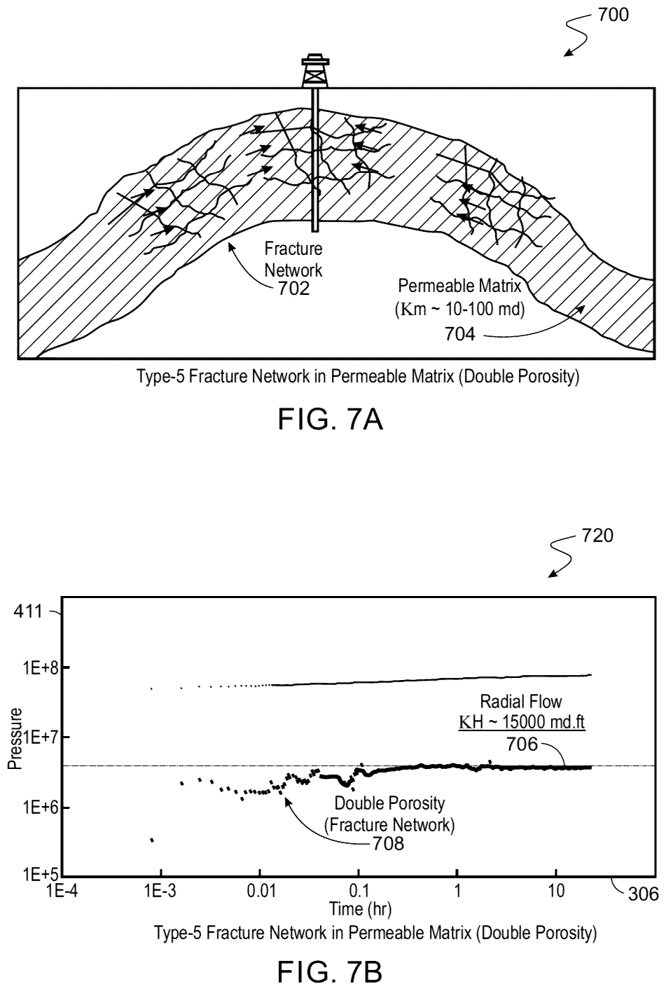

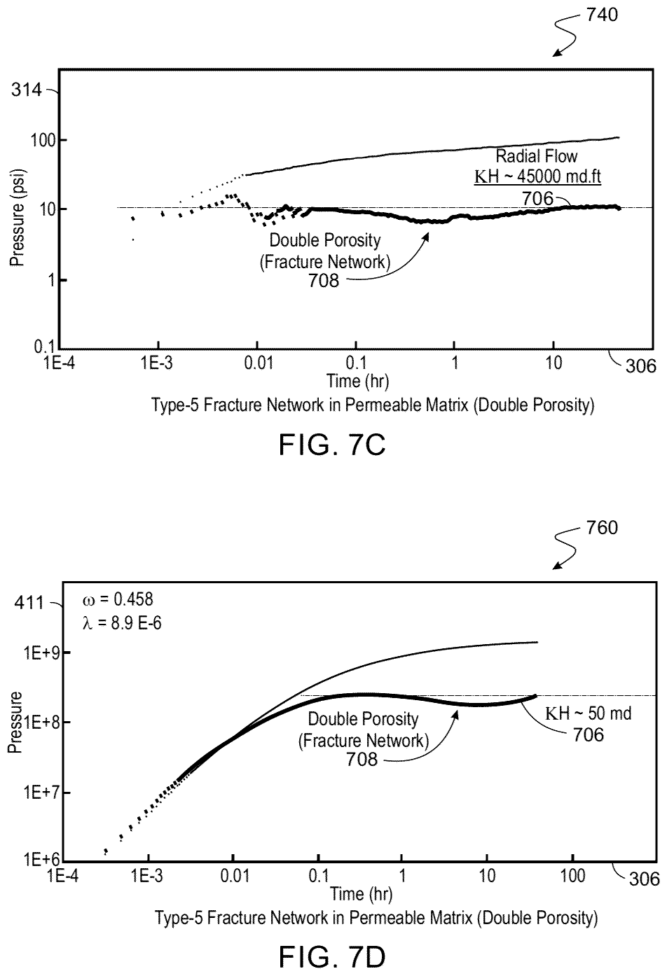

FIGS. 7A-7D collectively show an example of a dynamic response of Type-5 case of a fracture network in a permeable matrix (double porosity), according to some implementations of the present disclosure. FIGS. 7A-7D collectively show the dynamic response of Type-5 case: wells intersecting with fracture networks in permeable rocks (Double Porosity: Φ_{rock}+Φ_{fractures}). The fracture network is a connected cluster of discrete fractures. Wells drilled into these fracture clusters produce at sustainable high production rates and stabilized flowing pressure. The pressure derivative shape shows the “valley shape” in the early time followed by a horizontal line (radial flow). The difference between type-4 and type-5 dynamic responses is the description of fluid storativity inside the reservoir rocks. In type-4, the fluid volume is stored inside the fractures while the rock matrix has low fluid volumes. In type-5 both fractures and rock matrix contains a considerable portion of the fluid volume (rock storativity). The impact of distinguishing the difference between type-4 and type-5 is crucial in the drilling and production strategies of these reservoirs.

FIG. 7A shows a fracture network 702 in a permeable matrix 704 (e.g., Km in the range of about 10-100 md). FIG. 7B shows a KH 706 (e.g., approximately 15,000 md-ft) and a double porosity 708, plotted relative to time 306 and pressure 314. In FIG. 7C, KH 706 is approximately 45,000 md-ft. In FIG. 7D, KH 706 is approximately 50 md-ft.

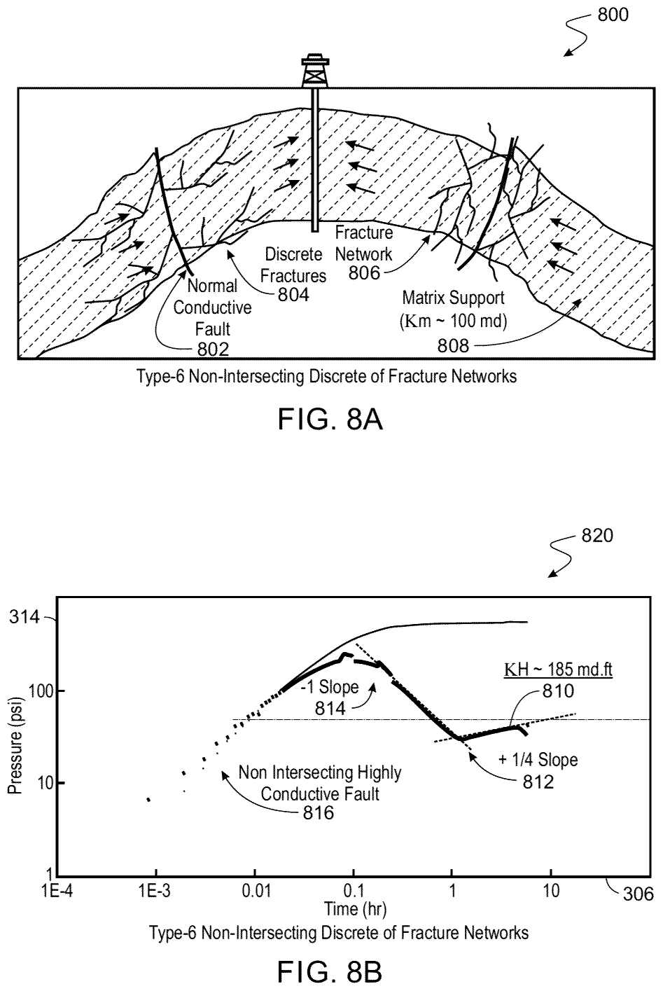

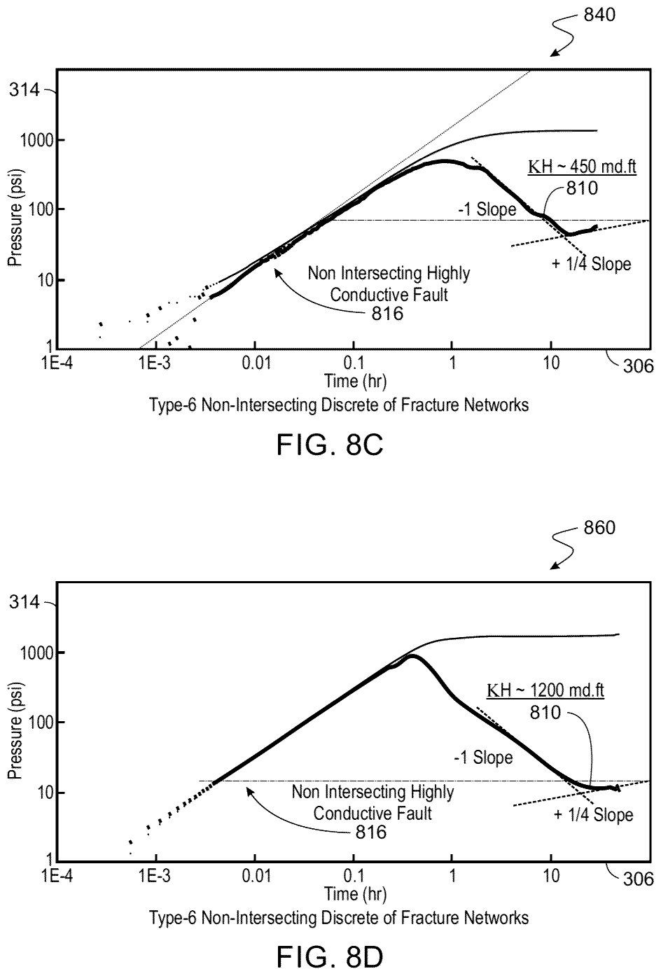

FIGS. 8A-SD collectively show an example of a dynamic response of Type-6 case of non-intersecting discrete fractures or networks, according to some implementations of the present disclosure. FIGS. 8A-8D collectively show the dynamic response of Type-6 case: wells drilled non-intersecting with conductive faults/fractures. These conductive faults can be easily mapped from seismic data due to its vertical and lateral extent with a recognizable throw. The collaboration of fault mapping with the geo-mechanical modeling of various stress regimes provides a static description of the faults conductive (ability to allow fluid migration across them) or sealing capacity (a barrier that prevents fluid movement). The static description of faults is verified by the dynamic description of well testing and pressure transient analysis. Wells drilled in the regions close to the conductive fault in high permeable rocks can produce massive production rates with very low drawdown due to the presence of small fissures that connects the well to the conductive faults. The infinite magnitude of conductivity of these regional fractures causes the flow inside the reservoir to be similar to a pipe flow rather than matrix flow. The derivative plot shows middle time fast drop with a slope of negative unit slope (−1) or negative half slope (−½) followed by a positive quarter slope (+¼).

The present disclosure introduces the newly recognized pressure derivative pattern of a well non-intersecting a highly conductive fault (fracture) that shows a negative unit slope (−1) followed by a positive quarter slope (+¼).

FIG. 8A shows a normal conductive fault 802 among discrete faults 804. FIG. 8A also shows a fracture network 806 and matrix support 808 (e.g., with Km approximately 100 md). FIG. 8B shows a KH 810 of approximately 185 md-ft. The graph has a slope 814 of −1 and a slope 812 of +1/4 for a non-intersecting highly conductive fault 816. In FIG. SC, the KH 810 of approximately 450 md-ft. In FIG. 8D, the KH 810 of approximately 1200 md-ft.

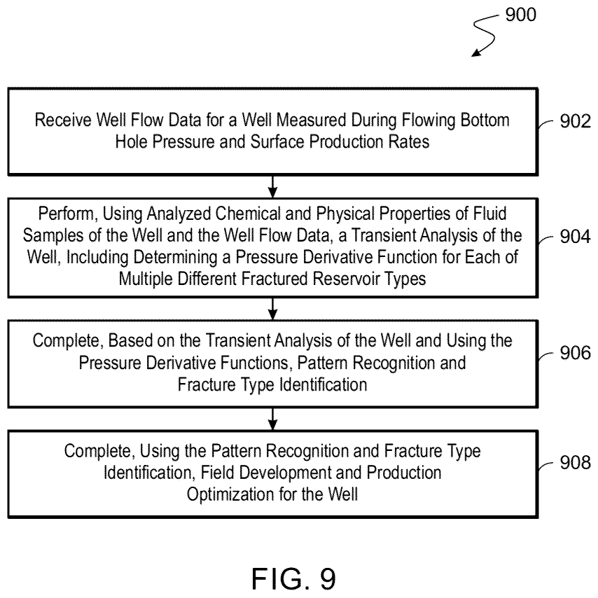

FIG. 9 is a flowchart of an example of a method 900 for identifying fractured reservoir types, according to some implementations of the present disclosure. For clarity of presentation, the description that follows generally describes method 900 in the context of the other figures in this description. However, it will be understood that method 900 can be performed, for example, by any suitable system, environment, software, and hardware, or a combination of systems, environments, software, and hardware, as appropriate. In some implementations, various steps of method 900 can be run in parallel, in combination, in loops, or in any order.

At 902, well flow data is received for a well measured during flowing bottom hole pressure and surface production rates. For example, measuring the well during flowing bottom hole pressure and surface production rates can include shutting-in the well while measuring shut-in bottom hole pressure for a time period that is twice a flowing time of the well. Method 900 can further include collecting fluid samples from the well and measuring and analyzing chemical and physical properties using the fluid samples. From 902, method 900 proceeds to 904.

At 904, a transient analysis of the well is performed using well flow data and analyzed chemical and physical properties of fluid samples of the well. A pressure derivative function is determined for each of multiple different fractured reservoir types. As an example, performing transient analysis of the well can include: collecting dynamic pressure data for the well; determining production rates with static fluid properties; determining the pressure derivative function for each of the multiple different fractured reservoir types; plotting, for each of the multiple different fractured reservoir types on a log-log scale, a pressure and pressure derivative function; and plotting, for each of the multiple different fractured reservoir types on a semi-log scale, a pressure and production time function. The multiple different fractured reservoir types include six different fractured reservoir types, for example, identified with reference to FIGS. 1E, 2A, and 3A-8D. From 904, method 900 proceeds to 906.

At 906, pattern recognition and fracture type identification are completed based on the transient analysis of the well and using the pressure derivative functions. As an example, completing the pattern recognition and fracture type identification can include: identifying, using the pressure derivative function for each of multiple different fractured reservoir types, linear segments related to fractures along a wellbore of the well; categorizing and typing the fractures by matching the pressure derivative function to fractured reservoir models using a type curve modelling method to determine fracture conductivity, geometry, and abundance; and correlating reservoir flow performance to the identified fracture types. From 906, method 900 proceeds to 908.

At 908, field development and production optimization for the well are completed using the pattern recognition and fracture type identification. As an example, completing the pattern recognition and fracture type identification can include: identifying locations of new wells and adjusting locations of producing wells to realize optimum production rates and to maximize hydrocarbon recovery; and adjusting, using at least the locations, well completion types and components to obtain desired reservoir flow patterns. After 908, method 900 can stop.

In some implementations, in addition to (or in combination with) any previously-described features, techniques of the present disclosure can include the following. Outputs of the techniques of the present disclosure can be performed before, during, or in combination with wellbore operations, such as to provide inputs to change the settings or parameters of equipment used for drilling. Examples of wellbore operations include forming/drilling a wellbore, hydraulic fracturing, and producing through the wellbore, to name a few. The wellbore operations can be triggered or controlled, for example, by outputs of the methods of the present disclosure. In some implementations, customized user interfaces can present intermediate or final results of the above described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or “app”), or at a central processing facility. The presented information can include suggestions, such as suggested changes in parameters or processing inputs, that the user can select to implement improvements in a production environment, such as in the exploration, production, and/or testing of petrochemical processes or facilities. For example, the suggestions can include parameters that, when selected by the user, can cause a change to, or an improvement in, drilling parameters (including drill bit speed and direction) or overall production of a gas or oil well. The suggestions, when implemented by the user, can improve the speed and accuracy of calculations, streamline processes, improve models, and solve problems related to efficiency, performance, safety, reliability, costs, downtime, and the need for human interaction. This can lead to (or be part of) an optimization of production or performance as indicated by an increase in performance greater than a predefined threshold. In some implementations, the suggestions can be implemented in real-time, such as to provide an immediate or near-immediate change in operations or in a model. The term real-time can correspond, for example, to events that occur within a specified period of time, such as within one minute or within one second. Events can include readings or measurements captured by downhole equipment such as sensors, pumps, bottom hole assemblies, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart, or are located in different countries or other jurisdictions.

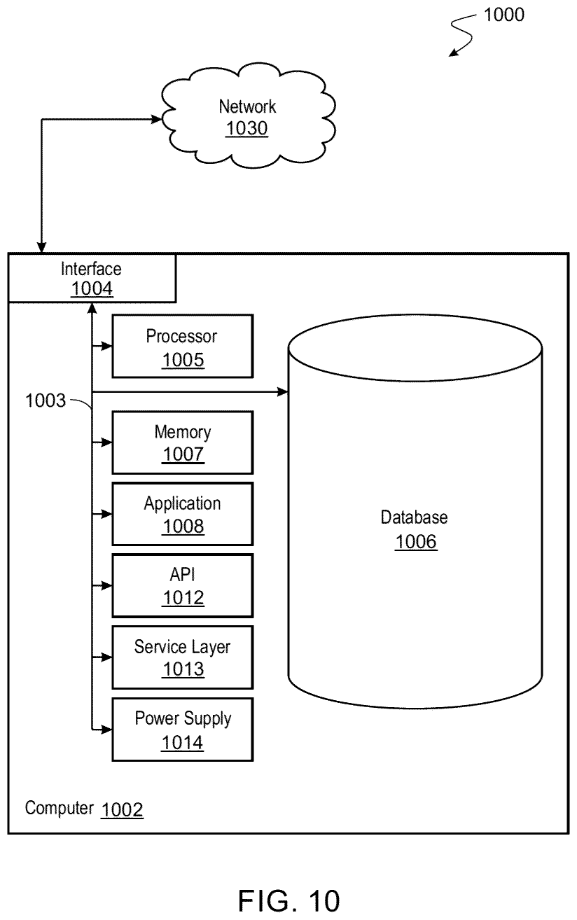

FIG. 10 is a block diagram of an example computer system 1000 used to provide computational functionalities associated with described algorithms, methods, functions, processes, flows, and procedures described in the present disclosure, according to some implementations of the present disclosure. The illustrated computer 1002 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 1002 can include input devices such as keypads, keyboards, and touch screens that can accept user information. Also, the computer 1002 can include output devices that can convey information associated with the operation of the computer 1002. The information can include digital data, visual data, audio information, or a combination of information. The information can be presented in a graphical user interface (UI) (or GUI).

The computer 1002 can serve in a role as a client, a network component, a server, a database, a persistency, or components of a computer system for performing the subject matter described in the present disclosure. The illustrated computer 1002 is communicably coupled with a network 1030. In some implementations, one or more components of the computer 1002 can be configured to operate within different environments, including cloud-computing-based environments, local environments, global environments, and combinations of environments.

At a top level, the computer 1002 is an electronic computing device operable to receive, transmit, process, store, and manage data and information associated with the described subject matter. According to some implementations, the computer 1002 can also include, or be communicably coupled with, an application server, an email server, a web server, a caching server, a streaming data server, or a combination of servers.

The computer 1002 can receive requests over network 1030 from a client application (for example, executing on another computer 1002). The computer 1002 can respond to the received requests by processing the received requests using software applications. Requests can also be sent to the computer 1002 from internal users (for example, from a command console), external (or third) parties, automated applications, entities, individuals, systems, and computers.

Each of the components of the computer 1002 can communicate using a system bus 1003. In some implementations, any or all of the components of the computer 1002, including hardware or software components, can interface with each other or the interface 1004 (or a combination of both) over the system bus 1003. Interfaces can use an application programming interface (API) 1012, a service layer 1013, or a combination of the API 1012 and service layer 1013. The API 1012 can include specifications for routines, data structures, and object classes. The API 1012 can be either computer-language independent or dependent. The API 1012 can refer to a complete interface, a single function, or a set of APIs.

The service layer 1013 can provide software services to the computer 1002 and other components (whether illustrated or not) that are communicably coupled to the computer 1002. The functionality of the computer 1002 can be accessible for all service consumers using this service layer. Software services, such as those provided by the service layer 1013, can provide reusable, defined functionalities through a defined interface. For example, the interface can be software written in JAVA, C++, or a language providing data in extensible markup language (XML) format. While illustrated as an integrated component of the computer 1002, in alternative implementations, the API 1012 or the service layer 1013 can be stand-alone components in relation to other components of the computer 1002 and other components communicably coupled to the computer 1002. Moreover, any or all parts of the API 1012 or the service layer 1013 can be implemented as child or sub-modules of another software module, enterprise application, or hardware module without departing from the scope of the present disclosure.

The computer 1002 includes an interface 1004. Although illustrated as a single interface 1004 in FIG. 10, two or more interfaces 1004 can be used according to particular needs, desires, or particular implementations of the computer 1002 and the described functionality. The interface 1004 can be used by the computer 1002 for communicating with other systems that are connected to the network 1030 (whether illustrated or not) in a distributed environment. Generally, the interface 1004 can include, or be implemented using, logic encoded in software or hardware (or a combination of software and hardware) operable to communicate with the network 1030. More specifically, the interface 1004 can include software supporting one or more communication protocols associated with communications. As such, the network 1030 or the interface's hardware can be operable to communicate physical signals within and outside of the illustrated computer 1002.

The computer 1002 includes a processor 1005. Although illustrated as a single processor 1005 in FIG. 10, two or more processors 1005 can be used according to particular needs, desires, or particular implementations of the computer 1002 and the described functionality. Generally, the processor 1005 can execute instructions and can manipulate data to perform the operations of the computer 1002, including operations using algorithms, methods, functions, processes, flows, and procedures as described in the present disclosure.

The computer 1002 also includes a database 1006 that can hold data for the computer 1002 and other components connected to the network 1030 (whether illustrated or not). For example, database 1006 can be an in-memory, conventional, or a database storing data consistent with the present disclosure. In some implementations, database 1006 can be a combination of two or more different database types (for example, hybrid in-memory and conventional databases) according to particular needs, desires, or particular implementations of the computer 1002 and the described functionality. Although illustrated as a single database 1006 in FIG. 10, two or more databases (of the same, different, or combination of types) can be used according to particular needs, desires, or particular implementations of the computer 1002 and the described functionality. While database 1006 is illustrated as an internal component of the computer 1002, in alternative implementations, database 1006 can be external to the computer 1002.

The computer 1002 also includes a memory 1007 that can hold data for the computer 1002 or a combination of components connected to the network 1030 (whether illustrated or not). Memory 1007 can store any data consistent with the present disclosure. In some implementations, memory 1007 can be a combination of two or more different types of memory (for example, a combination of semiconductor and magnetic storage) according to particular needs, desires, or particular implementations of the computer 1002 and the described functionality. Although illustrated as a single memory 1007 in FIG. 10, two or more memories 1007 (of the same, different, or combination of types) can be used according to particular needs, desires, or particular implementations of the computer 1002 and the described functionality. While memory 1007 is illustrated as an internal component of the computer 1002, in alternative implementations, memory 1007 can be external to the computer 1002.

The application 1008 can be an algorithmic software engine providing functionality according to particular needs, desires, or particular implementations of the computer 1002 and the described functionality. For example, application 1008 can serve as one or more components, modules, or applications. Further, although illustrated as a single application 1008, the application 1008 can be implemented as multiple applications 1008 on the computer 1002. In addition, although illustrated as internal to the computer 1002, in alternative implementations, the application 1008 can be external to the computer 1002.

The computer 1002 can also include a power supply 1014. The power supply 1014 can include a rechargeable or non-rechargeable battery that can be configured to be either user-or non-user-replaceable. In some implementations, the power supply 1014 can include power-conversion and management circuits, including recharging, standby, and power management functionalities. In some implementations, the power supply 1014 can include a power plug to allow the computer 1002 to be plugged into a wall socket or a power source to, for example, power the computer 1002 or recharge a rechargeable battery.

There can be any number of computers 1002 associated with, or external to, a computer system containing computer 1002, with each computer 1002 communicating over network 1030. Further, the terms “client,” “user,” and other appropriate terminology can be used interchangeably, as appropriate, without departing from the scope of the present disclosure. Moreover, the present disclosure contemplates that many users can use one computer 1002 and one user can use multiple computers 1002.

Described implementations of the subject matter can include one or more features. alone or in combination.

For example, in a first implementation, a computer-implemented method includes the following. Well flow data that is received for a well is measured during flowing bottom hole pressure and surface production rates. A transient analysis of the well is performed using the well flow data and analyzed chemical and physical properties of fluid samples of the well. A pressure derivative function is determined for each of multiple different fractured reservoir types. Pattern recognition and fracture type identification are completed based on the transient analysis of the well and using the pressure derivative functions. Field development and production optimization for the well are completed using the pattern recognition and fracture type identification.

The foregoing and other described implementations can each, optionally, include one or more of the following features:

A first feature, combinable with any of the following features, where the method further includes: collecting fluid samples from the well; and measuring and analyzing chemical and physical properties using the fluid samples.

A second feature, combinable with any of the previous or following features, where measuring the well during flowing bottom hole pressure and surface production rates includes shutting-in the well while measuring shut-in bottom hole pressure for a time period that is twice a flowing time of the well.

A third feature, combinable with any of the previous or following features, where performing transient analysis of the well includes: collecting dynamic pressure data for the well; determining production rates with static fluid properties; determining the pressure derivative function for each of the multiple different fractured reservoir types; plotting, for each of the multiple different fractured reservoir types on a log-log scale, a pressure and pressure derivative function; and plotting, for each of the multiple different fractured reservoir types on a semi-log scale, a pressure and production time function.

A fourth feature, combinable with any of the previous or following features, where the multiple different fractured reservoir types include six different fractured reservoir types.

A fifth feature, combinable with any of the previous or following features, where completing the pattern recognition and fracture type identification includes: identifying, using the pressure derivative function for each of multiple different fractured reservoir types, linear segments related to fractures along a wellbore of the well; categorizing and typing the fractures by matching the pressure derivative function to fractured reservoir models using a type curve modelling method to determine fracture conductivity, geometry, and abundance; and correlating reservoir flow performance to the identified fracture types.

A sixth feature, combinable with any of the previous or following features, where completing the pattern recognition and fracture type identification includes: identifying locations of new wells and adjusting locations of producing wells to realize optimum production rates and to maximize hydrocarbon recovery; and adjusting, using one or more of the locations of the new wells and the locations of the producing wells, well completion types and components to obtain desired reservoir flow patterns.

In a second implementation, a non-transitory, computer-readable medium stores one or more instructions executable by a computer system to perform operations including the following. Well flow data that is received for a well is measured during flowing bottom hole pressure and surface production rates. A transient analysis of the well is performed using the well flow data and analyzed chemical and physical properties of fluid samples of the well. A pressure derivative function is determined for each of multiple different fractured reservoir types. Pattern recognition and fracture type identification are completed based on the transient analysis of the well and using the pressure derivative functions. Field development and production optimization for the well are completed using the pattern recognition and fracture type identification.

The foregoing and other described implementations can each, optionally, include one or more of the following features:

A first feature, combinable with any of the following features, where the operations further include: collecting fluid samples from the well; and measuring and analyzing chemical and physical properties using the fluid samples.

A second feature, combinable with any of the previous or following features, where measuring the well during flowing bottom bole pressure and surface production rates includes shutting-in the well while measuring shut-in bottom hole pressure for a time period that is twice a flowing time of the well.

A third feature, combinable with any of the previous or following features, where performing transient analysis of the well includes: collecting dynamic pressure data for the well; determining production rates with static fluid properties; determining the pressure derivative function for each of the multiple different fractured reservoir types; plotting, for each of the multiple different fractured reservoir types on a log-log scale, a pressure and pressure derivative function; and plotting, for each of the multiple different fractured reservoir types on a semi-log scale, a pressure and production time function.

A fourth feature, combinable with any of the previous or following features, where the multiple different fractured reservoir types include six different fractured reservoir types.

A fifth feature, combinable with any of the previous or following features, where completing the pattern recognition and fracture type identification includes: identifying, using the pressure derivative function for each of multiple different fractured reservoir types, linear segments related to fractures along a wellbore of the well; categorizing and typing the fractures by matching the pressure derivative function to fractured reservoir models using a type curve modelling method to determine fracture conductivity, geometry, and abundance; and correlating reservoir flow performance to the identified fracture types.

A sixth feature, combinable with any of the previous or following features, where completing the pattern recognition and fracture type identification includes: identifying locations of new wells and adjusting locations of producing wells to realize optimum production rates and to maximize hydrocarbon recovery; and adjusting, using one or more of the locations of the new wells and the locations of the producing wells, well completion types and components to obtain desired reservoir flow patterns.

In a third implementation, a computer-implemented system includes one or more processors and a non-transitory computer-readable storage medium coupled to the one or more processors and storing programming instructions for execution by the one or more processors. The programming instructions instruct the one or more processors to perform operations including the following. Well flow data that is received for a well is measured during flowing bottom hole pressure and surface production rates. A transient analysis of the well is performed using the well flow data and analyzed chemical and physical properties of fluid samples of the well. A pressure derivative function is determined for each of multiple different fractured reservoir types. Pattern recognition and fracture type identification are completed based on the transient analysis of the well and using the pressure derivative functions. Field development and production optimization for the well are completed using the pattern recognition and fracture type identification.

The foregoing and other described implementations can each, optionally, include one or more of the following features:

A first feature, combinable with any of the following features, where the operations further include: collecting fluid samples from the well; and measuring and analyzing chemical and physical properties using the fluid samples.

A second feature, combinable with any of the previous or following features, where measuring the well during flowing bottom hole pressure and surface production rates includes shutting-in the well while measuring shut-in bottom hole pressure for a time period that is twice a flowing time of the well.

A third feature, combinable with any of the previous or following features, where performing transient analysis of the well includes: collecting dynamic pressure data for the well; determining production rates with static fluid properties; determining the pressure derivative function for each of the multiple different fractured reservoir types; plotting, for each of the multiple different fractured reservoir types on a log-log scale, a pressure and pressure derivative function: and plotting, for each of the multiple different fractured reservoir types on a semi-log scale, a pressure and production time function.

A fourth feature, combinable with any of the previous or following features, where the multiple different fractured reservoir types include six different fractured reservoir types.

A fifth feature, combinable with any of the previous or following features, where completing the pattern recognition and fracture type identification includes: identifying, using the pressure derivative function for each of multiple different fractured reservoir types, linear segments related to fractures along a wellbore of the well; categorizing and typing the fractures by matching the pressure derivative function to fractured reservoir models using a type curve modelling method to determine fracture conductivity, geometry, and abundance; and correlating reservoir flow performance to the identified fracture types.

A sixth feature, combinable with any of the previous or following features, where completing the pattern recognition and fracture type identification includes: identifying locations of new wells and adjusting locations of producing wells to realize optimum production rates and to maximize hydrocarbon recovery; and adjusting, using at least the locations, well completion types and components to obtain desired reservoir flow patterns.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to a suitable receiver apparatus for execution by a data processing apparatus. The computer-storage medium can be a machine-readable storage device, a machine-readable storage substrate, a random or serial access memory device, or a combination of computer-storage mediums.

The term “real-time,” “real time,” “realtime,” “real (fast) time (RFT),” “near(ly) real-time (NRT),” “quasi real-time,” or similar terms (as understood by one of ordinary skill in the art), means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second(s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

The terms “data processing apparatus,” “computer,” and “electronic computer device” (or equivalent as understood by one of ordinary skill in the art) refer to data processing hardware. For example, a data processing apparatus can encompass all kinds of apparatuses, devices, and machines for processing data, including by way of example, a programmable processor, a computer, or multiple processors or computers. The apparatus can also include special purpose logic circuitry including, for example, a central processing unit (CPU), a field-programmable gate array (FPGA), or an application-specific integrated circuit (ASIC). In some implementations, the data processing apparatus or special purpose logic circuitry (or a combination of the data processing apparatus or special purpose logic circuitry) can be hardware-or software-based (or a combination of both hardware-and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, such as LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub-programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory.

Graphics processing units (GPUS) can also be used in combination with CPUs. The GPUs can provide specialized processing that occurs in parallel to processing performed by CPUs. The specialized processing can include artificial intelligence (AI) applications and processing, for example. GPUs can be used in GPU clusters or in multi-GPU computing.

A computer can include, or be operatively coupled to, one or more mass storage devices for storing data. In some implementations, a computer can receive data from, and transfer data to, the mass storage devices including, for example, magnetic, magneto-optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer-readable media (transitory or non-transitory, as appropriate) suitable for storing computer program instructions and data can include all forms of permanent/non-permanent and volatile/non-volatile memory, media, and memory devices. Computer-readable media can include, for example, semiconductor memory devices such as random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer-readable media can also include, for example, magnetic devices such as tape, cartridges, cassettes, and internal/removable disks. Computer-readable media can also include magneto-optical disks and optical memory devices and technologies including, for example, digital video disc (DVD), CD-ROM, DVD+/−R, DVD-RAM, DVD-ROM, HD-DVD, and BLU-RAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated into, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that the user uses. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch-screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at the application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations. It should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system including a computer memory interoperably coupled with a hardware processor configured to perform the computer-implemented method or the instructions sored on the non-transitory, computer-readable medium.

What is claimed is:

1. A computer-implemented method, comprising:
receiving well flow data for a well measured during flowing bottom hole pressure and surface production rates;
performing, using analyzed chemical and physical properties of fluid samples of the well and the well flow data, a transient analysis of the well, including determining a pressure derivative function for each of multiple different fractured reservoir types;
completing, based on the transient analysis of the well and using the pressure derivative functions, pattern recognition and fracture type identification; and
completing, using the pattern recognition and fracture type identification, field development and production optimization for the well.

2. The computer-implemented method of claim 1, further comprising:
collecting fluid samples from the well; and
measuring and analyzing chemical and physical properties using the fluid samples.

3. The computer-implemented method of claim 1, wherein measuring the well during flowing bottom hole pressure and surface production rates includes shutting-in the well while measuring shut-in bottom hole pressure for a time period that is twice a flowing time of the well.

4. The computer-implemented method of claim 1, wherein performing transient analysis of the well includes:
collecting dynamic pressure data for the well;
determining production rates with static fluid properties;
determining the pressure derivative function for each of the multiple different fractured reservoir types;
plotting, for each of the multiple different fractured reservoir types on a log-log scale, a pressure and pressure derivative function; and
plotting, for each of the multiple different fractured reservoir types on a semi-log scale, a pressure and production time function.

5. The computer-implemented method of claim 1, wherein the multiple different fractured reservoir types include six different fractured reservoir types.

6. The computer-implemented method of claim 1, wherein completing the pattern recognition and fracture type identification includes:
identifying, using the pressure derivative function for each of multiple different fractured reservoir types, linear segments related to fractures along a wellbore of the well;
categorizing and typing the fractures by matching the pressure derivative function to fractured reservoir models using a type curve modelling method to determine fracture conductivity, geometry, and abundance; and
correlating reservoir flow performance to the identified fracture types.

7. The computer-implemented method of claim 1, wherein completing the pattern recognition and fracture type identification includes:
identifying locations of new wells and adjusting locations of producing wells to realize optimum production rates and to maximize hydrocarbon recovery; and
adjusting, using one or more of the locations of the new wells and the locations of the producing wells, well completion types and components to obtain desired reservoir flow patterns.

8. A non-transitory, computer-readable medium storing one or more instructions executable by a computer system to perform operations comprising:
receiving well flow data for a well measured during flowing bottom hole pressure and surface production rates;
performing, using analyzed chemical and physical properties of fluid samples of the well and the well flow data, a transient analysis of the well, including determining a pressure derivative function for each of multiple different fractured reservoir types;
completing, based on the transient analysis of the well and using the pressure derivative functions, pattern recognition and fracture type identification; and
completing, using the pattern recognition and fracture type identification, field development and production optimization for the well.

9. The non-transitory, computer-readable medium of claim 8, the operations further comprising:
collecting fluid samples from the well; and
measuring and analyzing chemical and physical properties using the fluid samples.

10. The non-transitory, computer-readable medium of claim 8, wherein measuring the well during flowing bottom hole pressure and surface production rates includes shutting-in the well while measuring shut-in bottom hole pressure for a time period that is twice a flowing time of the well.

11. The non-transitory, computer-readable medium of claim 8, wherein performing transient analysis of the well includes:
collecting dynamic pressure data for the well;
determining production rates with static fluid properties;
determining the pressure derivative function for each of the multiple different fractured reservoir types;
plotting, for each of the multiple different fractured reservoir types on a log-log scale, a pressure and pressure derivative function; and
plotting, for each of the multiple different fractured reservoir types on a semi-log scale, a pressure and production time function.

12. The non-transitory, computer-readable medium of claim 8, wherein the multiple different fractured reservoir types include six different fractured reservoir types.

13. The non-transitory, computer-readable medium of claim 8, wherein completing the pattern recognition and fracture type identification includes:
identifying, using the pressure derivative function for each of multiple different fractured reservoir types, linear segments related to fractures along a wellbore of the well;
categorizing and typing the fractures by matching the pressure derivative function to fractured reservoir models using a type curve modelling method to determine fracture conductivity, geometry, and abundance; and
correlating reservoir flow performance to the identified fracture types.

14. The non-transitory, computer-readable medium of claim 8, wherein completing the pattern recognition and fracture type identification includes:
identifying locations of new wells and adjusting locations of producing wells to realize optimum production rates and to maximize hydrocarbon recovery; and
adjusting, using one or more of the locations of the new wells and the locations of the producing wells, well completion types and components to obtain desired reservoir flow patterns.

15. A computer-implemented system, comprising:
one or more processors; and
a non-transitory computer-readable storage medium coupled to the one or more processors and storing programming instructions for execution by the one or more processors, the programming instructions instructing the one or more processors to perform operations comprising:
receiving well flow data for a well measured during flowing bottom hole pressure and surface production rates;
performing, using analyzed chemical and physical properties of fluid samples of the well and the well flow data, a transient analysis of the well, including determining a pressure derivative function for each of multiple different fractured reservoir types;
completing, based on the transient analysis of the well and using the pressure derivative functions, pattern recognition and fracture type identification; and
completing, using the pattern recognition and fracture type identification, field development and production optimization for the well.

16. The computer-implemented system of claim 15, the operations further comprising:
collecting fluid samples from the well; and
measuring and analyzing chemical and physical properties using the fluid samples.

17. The computer-implemented system of claim 15, wherein measuring the well during flowing bottom hole pressure and surface production rates includes shutting-in the well while measuring shut-in bottom hole pressure for a time period that is twice a flowing time of the well.

18. The computer-implemented system of claim 15, wherein performing transient analysis of the well includes:
collecting dynamic pressure data for the well;
determining production rates with static fluid properties;
determining the pressure derivative function for each of the multiple different fractured reservoir types;
plotting, for each of the multiple different fractured reservoir types on a log-log scale, a pressure and pressure derivative function; and
plotting, for each of the multiple different fractured reservoir types on a semi-log scale, a pressure and production time function.

19. The computer-implemented system of claim 15, wherein the multiple different fractured reservoir types include six different fractured reservoir types.

20. The computer-implemented system of claim 15, wherein completing the pattern recognition and fracture type identification includes:
identifying, using the pressure derivative function for each of multiple different fractured reservoir types, linear segments related to fractures along a wellbore of the well;
categorizing and typing the fractures by matching the pressure derivative function to fractured reservoir models using a type curve modelling method to determine fracture conductivity, geometry, and abundance; and
correlating reservoir flow performance to the identified fracture types.
